(19) United States
(12) Patent Application Publication
(10) Pub. No.: US 2006/0020267 A1
(43) Pub. Date: Jan. 26, 2006

(54) Intrastromal devices and methods for improving vision

(76) Inventor: J. Christopher Marmo, Danville, CA (US)

Correspondence Address: STOUT, UXA, BUYAN & MULLINS LLP, 4 VENTURE, SUITE 300, IRVINE, CA, 92618, US

(21) Appl. No.: 11/183,191

(22) Filed: Jul. 14, 2005

Related U.S. Application Data
(60) Provisional application No. 60/588,287, filed on Jul. 15, 2004.

(51) Int. Cl. A61F 9/013

(52) U.S. Cl. 606/107

(57) ABSTRACT

Methods for improving or correcting a patient's vision are provided which generally include inserting a lens within the stroma of an eye of the patient by forming an incision in the cornea and creating a pocket within the stroma for accommodating the lens, the method being accomplished without forming a flap incision.

### CROSS-REFERENCE TO RELATED APPLICATIONS

This application claims the benefit of U.S. Provisional Application No. 60/588,287, filed Jul. 15, 2004, the disclosure of which is hereby incorporated by reference in its entirety.

### BACKGROUND OF THE INVENTION

1. Field of the Invention

The present invention relates to devices and methods of enhancing an individual's vision. In particular, the invention relates to vision enhancing ocular devices and to methods comprising placing such devices within a stroma of the individual's eye. The ocular devices may be lenses, such as corneal inlays.

2. Description of Related Art

The cornea of the human eye provides between approximately 60 and 70 percent of the focusing power of the eye. As understood in the art, lenses may be placed in proximity of the cornea to augment the focusing capabilities of the eye. Examples of vision correction lenses include corneal inlays, which are implanted within the cornea, such as within the stroma of the cornea, corneal onlays, which are placed over the cornea after the epithelium has been removed, and contact lenses, which are placed over the corneal epithelium.

Procedures have been proposed for correcting vision using corneal inlay lenses which include forming a stromal flap of the cornea, opening the flap, exposing a portion of the stroma, placing a corrective lens onto the exposed stroma and repositioning the flap to cover the lens.

LASIK procedures using intra stromal inlay lenses permanently reshape the stroma of the eye in order to improve vision.

Stromal flaps formed during these surgical procedures are substantial in size and present significant risks and problems. For example, lenses, such as corneal inlays, placed beneath the flap often become decentered. In other circumstances, the flap may be prone to being displaced in the event of even minor physical trauma to the eye.

Thus, there remains a need for improved methods of improving vision with ocular devices placed in a corneal stroma.

### SUMMARY

Accordingly, methods are provided for improving, enhancing, or correcting vision which do not involve formation of a stromal flap. A method of the present invention comprises forming a pocket in the stroma of an eye of an individual.

In one embodiment, a method of enhancing vision of an individual comprises inserting an ocular device into a pocket formed in the stroma of the individual's eye. The ocular device may be a lens, and thus, the ocular device may be understood to be a corneal inlay. The pocket may be formed by forming an incision in the individual's cornea. The incision may be made at a nasal location, temporal location, superior location, inferior location, or combinations thereof. The incision may have a length from about 1 mm to about 6 mm in certain embodiments. The incision may be formed using a sharp cutting instrument, blunt dissection, or a combination thereof. The pocket is sized to accommodate an ocular device while reducing the possibility of the ocular device becoming decentered.

U.S. patent application Ser. Nos. 10/661,400, filed on Sep. 12, 2003, and 60/573,657, filed May 20, 2004, may contain information that is at least helpful or useful in understanding the present invention, and the entire disclosure each of the applications is incorporated herein by reference.

Any feature or combination of features described herein are included within the scope of the present invention provided that the features included in any such combination are not mutually inconsistent as will be apparent from the context, this specification, and the knowledge of one of ordinary skill in the art. In addition, any feature or combination of features may be specifically excluded from any embodiment of the present invention.

### BRIEF DESCRIPTION OF THE DRAWINGS

FIG. 1 is a diagram of a sectional view of a human eye.

FIG. 2 is a diagram of a magnified sectional view of the cornea of the human eye of FIG. 1.

FIG. 3 is a diagram of a front plan view of a corneal inlay lens to be implanted in an eye using methods of the present invention.

FIG. 4A is an illustration of a front plan view of an eye in which corneal tissue is formed as a stromal flap prior to placement of a corrective lens, in accordance with typical PRIOR ART methods.

FIG. 4B is a sectional view of the eye of FIG. 4A.

FIG. 4C is a sectional view similar to FIG. 4B in which the stromal flap has been replaced onto the eye after the lens has been placed on the stromal tissue.

FIG. 5A is an illustration of a front plan view of an eye in which an incision has been made and a pocket has been formed in the stroma in accordance with a method of the present invention.

FIG. 5B is a sectional view of the eye of FIG. 5A.

FIG. 5C is a sectional view similar to FIG. 5B in which a lens has been placed into the pocket, in accordance with a method of the present invention.

FIG. 6A is an illustration of a front plan view of an eye with a relatively large incision, however, the incision is not sufficiently large so as to form a stromal flap.

FIG. 6B is similar to FIG. 6A with a relatively smaller incision.

FIG. 6C is similar to FIG. 6B with an even smaller incision.

FIG. 7A is an illustration of a front plan view of an eye with an inferior incision.

FIG. 7B is a view similar to FIG. 7A showing an eye with a nasal incision.

FIG. 7C is a view similar to FIG. 7A showing an eye with a superior incision.

FIG. 8 is a simplified representation of a device being used to from a pocket by introducing a liquid into an initial incision, in accordance with a method of the present invention.

FIG. 9A is an illustration of a perspective view of a folded lens in which the lens is rolled prior to insertion into a stromal pocket incision in accordance with a method of the present invention.

FIG. 9B is an illustration of a perspective view of a folded lens in which the lens is folded along its midline prior to insertion into a stromal pocket incision in accordance with a method of the present invention.

### DESCRIPTION OF THE INVENTION

The present invention is generally directed to methods for enhancing, e.g., correcting, the vision of an individual, e.g., human or animal, by placing an ocular device within the stroma of a cornea of the patient. Preferably, the invention relates to methods including forming a stromal or intrastromal pocket in a cornea of an individual's eye, and placing or inserting a vision enhancing lens, e.g., a corneal inlay, into the pocket.

Methods for enhancing an individual's vision in accordance with the present invention generally include placing an ocular device, for example, a lens, within the stroma of the patient by forming a relatively small incision in the cornea. The incision advantageously penetrates the epithelium, Bowman's membrane and, usually but not always and not necessarily, stromal tissue, and creates a pocket within the stroma for accommodating the ocular device. The present methods may also include forming more than one pocket in the stroma, and inserting more than one ocular device into the stroma.

As illustrated in FIG. 1, a typical human eye 10 has a lens 12 and an iris 14. Posterior chamber 16 is located posterior to iris 14 and anterior chamber 18 is located anterior to iris 14. Eye 10 has a cornea 20 that consists of five layers, as discussed herein. One of the layers, corneal epithelium 22, forms the anterior exterior surface of cornea 20. Corneal epithelium 22 is a stratified squamous epithelium that extends laterally to the limbus 32. At limbus 32, corneal epithelium 22 becomes thicker and less regular to define the conjunctiva 34.

FIG. 2 illustrates a magnified view of the five layers of cornea 20. Typically, cornea 20 comprises corneal epithelium 22, Bowman's membrane 24, stroma 26, Descemet's membrane 28, and endothelium 30.

Corneal epithelium 22 usually is about 5-6 cell layers thick (approximately 50 micrometers thick), and generally regenerates when the cornea is injured. Corneal epithelium 22 provides a relatively smooth refractive surface and helps prevent infection of the eye. Bowman's membrane 24 lies between epithelium 22 and the stroma 26 and is believed to protect the cornea from injury.

Corneal stroma 26 is a laminated structure of collagen which contains cells, such as fibroblasts and keratocytes, dispersed therein. Stroma 26 constitutes about 90% of the corneal thickness.

Corneal endothelium 30 typically is a monolayer of low cuboidal or squamous cells that dehydrates the cornea by removing water from the cornea. An adult human cornea is typically about 500 μm (0.5 mm) thick and is typically devoid of blood vessels.

Limbus 32, shown in FIG. 1, is a region of transitions where cornea becomes sclera, and conjunctiva.

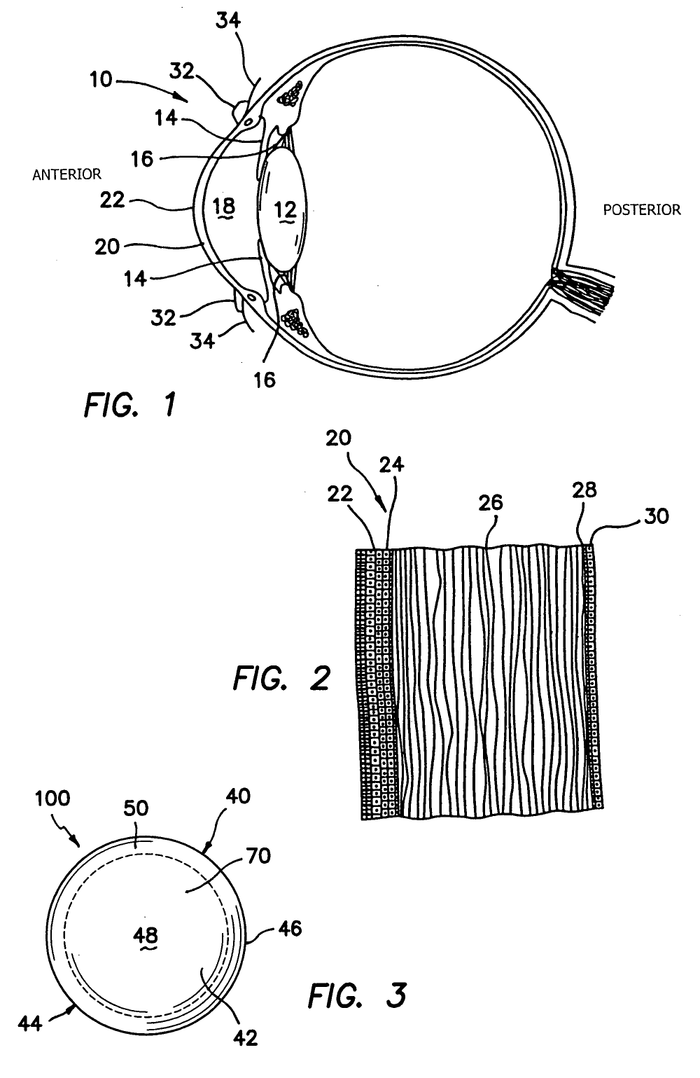

Turning to FIG. 3, an ocular device 100 for correcting, enhancing, or improving vision of an individual is shown. The device 100 is structured to alter the focusing capabilities of an individual's, such as a human patient's, eye, and preferably, the device 100 is structured to improve or enhance vision of a patient, for example, relative to the vision of the patient without the ocular device placed in the cornea of the individual. The device 100 is intended to be placed within the stroma of the cornea of an eye, and accordingly, device 100 may hereinafter sometimes be more specifically referred to as a corneal inlay lens or corneal inlay 40.

The lens 40 to be implanted in an eye in accordance with the methods of the present invention may have an optical power, including a predetermined optical power. The lens may be made from a hydrogel or non-hydrogel material suitable for vision correction. The lens may include a hydrogel portion or component and a non-hydrogel portion or component. The lens may have a substantially uniform composition or may be a composite, for example, having a layered configuration. The lens may comprise a synthetic material, for example a polymeric synthetic material. The lens may comprise collagen, including recombinant collagen.

The lens 40 may be fabricated from any suitable material or combination of materials that provide an optically clear lens to permit light to be transmitted to the retina of the eye when the lens is placed in the stromal pocket formed in the eye without substantially or unduly compromising the ocular physiology of the eye.

Generally, lens 40 has an anterior surface 42, a posterior surface 44, a peripheral edge 46 disposed at the juncture of anterior surface 42 and posterior surface 44. Anterior surface 42 is typically convex and posterior surface 44 is typically concave, however, the posterior surface may also include one or more planar portions or surfaces, or may be substantially planar. Lens 40 may also include an optic zone 48 and a peripheral zone 50. Typically, optic zone 48 is bounded by peripheral zone 50. Optic zone 48 is advantageously centrally located about an optical axis, such as a central optical axis, of the lens and peripheral zone 50 is disposed between an edge of optic zone 48 and peripheral edge 46.

Additional zones and lens configurations may be provided with the lens depending on the particular visual deficiency experienced by the patient and/or the particular visual deficiency of the patient to be addressed by the lens. Further, the lens may by substantially junctionless, that is, smooth and continuous, such that the lens has no areas or zones that have a visually or optically detectable junction.

The lens 40 may be optically configured to correct not only refractive errors, but also other optic aberrations of the eye and/or the optical device independently or in combination with correcting refractive errors. As understood by persons skilled in the art, lens 40 may be structured to correct visual deficiencies including, and not limited to, myopia, hyperopia, astigmatism, presbyopia and the like and combinations thereof. The lens 40 may correct or improve visual deficiencies by either optical means or physical means imposed on the stroma of the eye, or combinations thereof. Thus, the lens 40 may be a monofocal lens or a multifocal lens, including, without limitation, a bifocal lens. In addition, or alternatively, the lens 40 may be a toric lens. For example, the lens 40 may include a toric region which may be effective when placed on an eye with an astigmatism to correct or reduce the effects of the astigmatism.

For example, the lens 40 may include a toric region located on the posterior surface 44 of the lens 40, or the lens 40 may include a toric region located on the anterior surface 42. Advantageously, toric lenses may be used without requiring a ballast to maintain proper orientation of the lens on the eye since the lens may be held in a relatively fixed position within the stromal tissue. However, a ballast may be provided if desired. In certain embodiments, the lens 40 may include a ballast, such as a prism, or it may include one or more thinned regions, such as one or more inferior and/or superior thin zones. Ballasts may be helpful in maintaining the inlay in a fixed position, such as a fixed rotation position, in the stroma. Thus, a ballast may be understood to be an embodiment of a positioning member of the lens. The lens 40 may also include other positioning members or markings which may be helpful in aligning and/or maintaining the lens in a desired position when placed in the stroma of an eye.

In lenses configured to correct presbyobia, the lens may include one or more designs, such as concentric, aspheric (either with positive and/or negative spherical aberration), diffractive, and/or multi-zone refractive.

The lens 40 may have an optical power ranging from about −10.00 diopters to about +10.00 diopters, although other optical powers may be provided, and such other optical powers are within the scope of the present invention. Typically, a lens 40 will have a diameter in a range of about 2 mm to about 12 mm, for example, about 6 mm. The optic zone of the lens typically ranges from about 2 mm to about 10 mm, and preferably ranges from about 4 mm to about 8 mm, in diameter. The optic zone may be provided on either the anterior or posterior surface of the lens.

Lens 40 may comprise synthetic materials, non-synthetic materials, and combinations thereof. Lens 40 may comprise collagen, such as purified collagen. The collagen may be collagen Type I, which is the type of collagen that defines the bulk of the corneal stroma, or lens 40 may comprise one or more other types of collagen, including combinations of other types of collagen, such as Types III, IV, V, and VII, for example, with or without Type I collagen. In certain embodiments, the collagen may be obtained from animals, and/or humans. For example, collagen of the lens 40 may be bovine collagen, porcine collagen, avian collagen, murine collagen, equine collagen, among others and the like and combinations thereof.

Many different types of collagen useful in the lenses of the present invention are publicly available from companies, such as Becton Dickenson. In other embodiments, the collagen may be recombinantly synthesized, such as by using recombinant DNA technology. Preferably, lens 40 is not obtained from a donor patient, such as from corneal tissue of another individual person.

Collagen may be obtained using any conventional technique, as is practiced in the art. One source of publicly available recombinant collagen is FibroGen, South San Francisco, Calif. Alternatively, or in addition, recombinant collagen may be prepared and obtained using the methods disclosed in PCT Publication No. WO 93/07889 or WO 94/16570. The recombinant production techniques described in these PCT publications may readily be adapted so as to produce many different types of collagens, human and/or non-human. Utilizing purified collagen simplifies procedures of making the lens 40, as compared to a lens that is made of material obtained from donor tissue, such as disclosed in PCT Publication No. WO 02/06883.

Alternatively, lens 40 may be manufactured by obtaining and culturing corneal keratocytes, as disclosed in PCT Publication No. WO 99/37752 and U.S. Pat. No. 5,827,641. The cultures of keratocytes may be advantageously placed in a mold suitable for a vision correction lens, and produce a collagen matrix similar to a normal stroma in vivo. The various molds thus produce a corneal appliance having a synthetic stroma with a desired optical power to correct a vision deficiency of the patient.

Lens 40 may be made from a polymeric hydrogel, as understood by persons of ordinary skill in the art. A polymeric hydrogel includes a hydrogel-forming polymer, such as a water swellable polymer. The hydrogel itself includes such a polymer swollen with water. Polymeric hydrogels useful in the present invention, may have about 30% to about 80% by weight water, but may have about 20% to about 90% by weight water, or about 5% to about 95% by weight water, and advantageously have refractive indices in a range of about 1.3 to about 1.5, for example about 1.4, which is similar to the refractive indices of water and a human cornea.

Examples of suitable hydrogel-forming polymer materials or components of the lenses include, without limitation, poly(2-hydroxyethyl methacrylate) PHEMA, poly(glycerol methacrylate) PGMA, polyelectrolyte materials, polyethylene oxide, polyvinyl alcohol, polydioxaline, poly(acrylic acid), poly(acrylamide), poly(N-vinyl pyrilidone) and the like and mixtures thereof. Many of such materials are publicly available. In addition, one or more monomers which do not themselves produce homopolymers which are not hydrogel-forming polymers, such as methylmethacrylate (MMA), other methacrylates, acrylates and the like and mixtures thereof, can also be included in such hydrogel-forming polymer materials provided that the presence of units from such monomers does not interfere with the desired formation of a polymeric hydrogel.

Alternatively, and in certain embodiments, lens 40 may be manufactured to include a biocompatible, non-hydrogel material or component, such as disclosed in U.S. Pat. No. 5,713,957. Examples of non-hydrogel materials which may be included in the present ocular devices include, and are not limited to, acrylics, polyolefins, fluoropolymers, silicones, styrenics, vinyls, polyesters, polyurethanes, polycarbonates, cellulosics, proteins including collagen based materials and the like and combinations thereof. Furthermore, lens 40 may comprise a cell growth substrate polymer, such as those disclosed in U.S. Pat. No. 5,994,133.

Lens 40 may be made partly or substantially entirely from a synthetic material or from a combination of collagen and a synthetic material, including without limitation, combinations of bovine collagen and a synthetic material, and combinations of recombinant collagen and synthetic materials. Lens 40 may include a poly(N-isopropylacrylamide) (polynipam) component.

The lens 40 may include elements including physical perturbations of the lens 40, such as indentations provided in anterior surface 42 that facilitate attachment and do not alter the optical properties of the lens. Indentations may include pores that extend through the lens from the anterior surface to the posterior surface of the lens. The indentations may be provided over the entire lens or over only a fraction of the lens. The indentations may also be provided in specific patterns and/or specific dimensions that facilitate the functionality of the lens, for example, the attachment of the lens to the stroma, the compatibility of the lens in the stroma and the like. For example, the indentations may be provided in a plurality of concentric rings emanating from the center of the lens and expanding radially outward. The indentations may also be useful as markings to help position the lens in the proper orientation in the stroma.

As indicated above, lens 40 may include collagen to mimic a native corneal stroma, a hydrogel, a biocompatible non-hydrogel material and the like and combinations thereof. The lens 40 may be produced according to standard techniques known to those skilled in the art. As indicated above, when lenses for inclusion in the stroma are desired, a collagen matrix including stroma cells may be formed. Lens 40 may be shaped in a conventionally dimensioned mold suitable for forming lenses. For example, lens 40 may be ablated, molded, spin-casted and/or lathed, or combinations thereof. The mold may comprise a concave surface and a convex surface matingly shaped with respect to each other.

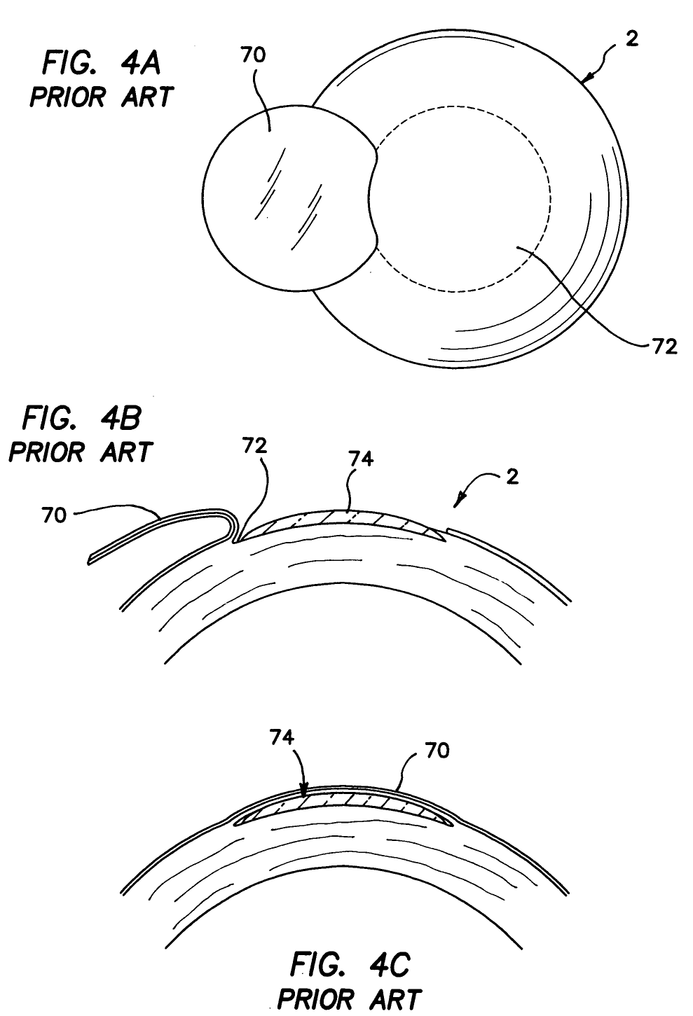

Turning now to FIGS. 4A-4C, a PRIOR ART method of correcting vision is shown. Generally, the method involves the creation of a relatively large stromal flap 70 of corneal tissue of an eye 2, typically using a microkeratome. As shown in FIG. 4A, the flap 70, which includes corneal epithelium, Bowman's membrane, and stromal tissue, is lifted to expose a bed of stromal tissue 72. Usually, the stromal bed is reshaped. For example, once exposed, portions of the stromal tissue may be removed or altered to a prescribed depth using an excimer laser. FIG. 4B illustrates a corrective lens 74 positioned on the exposed stromal tissue bed 72. FIG. 4C illustrates the eye 2 after the flap 70 has been repositioned and allowed to heal.

Unfortunately, the creation of a stromal flap using a microkeratome can result in some complications. Complications can result if the flap is cut improperly or completely severed from the cornea. Additional drawbacks associated with creating a flap include the inability to control the shape of the flap and the fact that a relatively large amount of corneal tissue needs to be cut to create the flap. In addition, the flap of tissue is much larger than the corneal inlay. The difference in size between the flap and the corneal inlay results in the inlay becoming decentered, which reduces or prevents the inlay from correcting a patient's vision.

In accordance with the present invention, improved methods are provided for correcting or enhancing vision that do not involve the creation of a stromal flap or any other substantial exposure of stromal tissue.

A method, in accordance with the present invention, for enhancing a patient's vision comprises placing or passing a vision correcting ocular device into a pocket formed in the stroma of an eye. Advantageously, the pocket requires a substantially smaller incision than that required to form a stromal flap. The diameter or length of the incision is advantageously substantially equal to, and preferably less than the diameter of the lens to be inserted into the pocket. For example, the incision may have a length less than about 12 mm, and in certain embodiments, the incision may have a length in a range of about 1 mm to about 6 mm or about 10 mm.

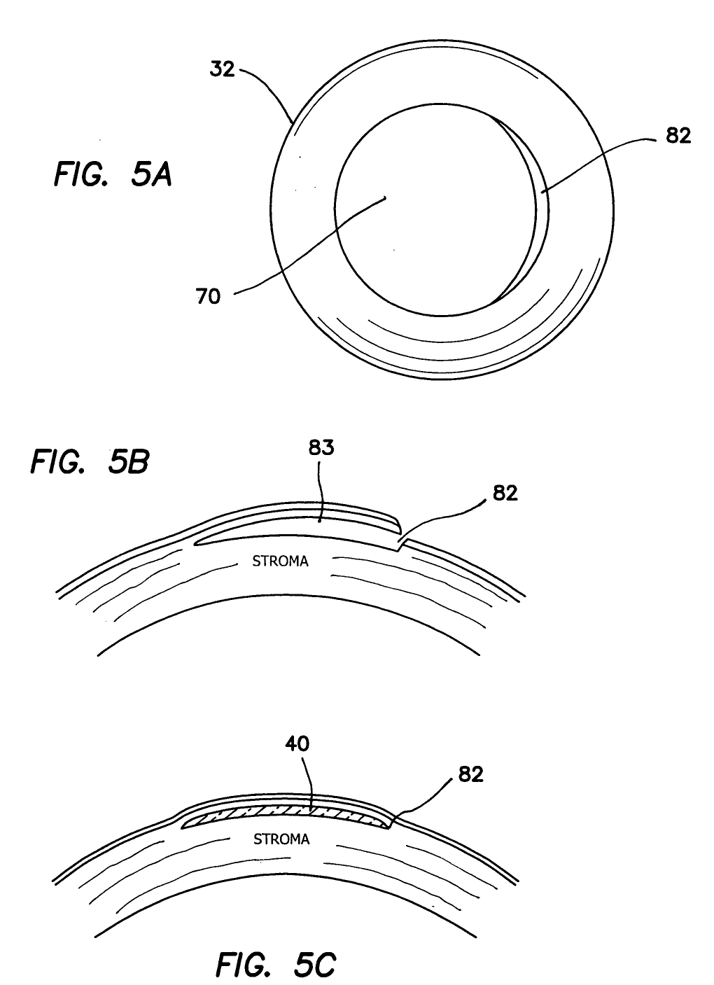

Turning to FIG. 5A, the method may comprise, for example, creating an incision, for example, a slit 82 or other relatively small opening, in the cornea. The incision is sufficiently large to permit a lens, in a flat, rolled, folded, coiled or other deformed configuration, to be passed therethrough.

After the initial slit 82 is formed, a pocket 83, shown in FIG. 5B, may be formed between adjacent layers of the stroma (or in other instances, between Bowman's membrane and the stroma), by gently separating these structures using standard blunt dissection techniques or other conventional methodology to form pocket 83 having a size and/or shape suitable for accommodating the lens. For example, the pocket may have a surface area substantially equal to or corresponding to a surface area of the lens. The lens 40, which may or may not be surface treated, may be passed or inserted into the pocket 83 as shown in FIG. 5C. After the lens is in position, a healing agent may be applied to the incision or to the eye to promote the healing thereof.

In accordance with other embodiments of the present invention, methods of correcting or enhancing vision are provided which generally include placing or inserting a vision correcting ocular device, for example, a corrective lens or lens body, between stromal layers of a patient's cornea substantially without uncovering or exposing tissues of the anterior structures of the cornea beneath the epithelium, wherein the anterior structures include Bowman's membrane and portions or layers of the corneal stroma.

The methods in accordance with the present invention thus are in contrast to prior art techniques that produce a flap of epithelial and stromal tissue to expose or uncover an anterior surface of the cornea, as discussed herein with reference to FIGS. 4A-4C. By placing an ocular device beneath an epithelium and Bowman's membrane, the ocular device is effectively substantially fixedly positioned with respect to the eye, for example, by the epithelium and Bowman's membrane and anterior layers of the stroma, to provide the desired vision correction. For example, by forming a pocket to have a diameter substantially equal to the diameter of an unfolded lens or inlay, decentration of the inlay is reduced and is preferably avoided. In addition, the present methods provide for relatively enhanced healing or reduced times and reduced side effects relative to methods that produce a flap of tissue to insert an ocular device.

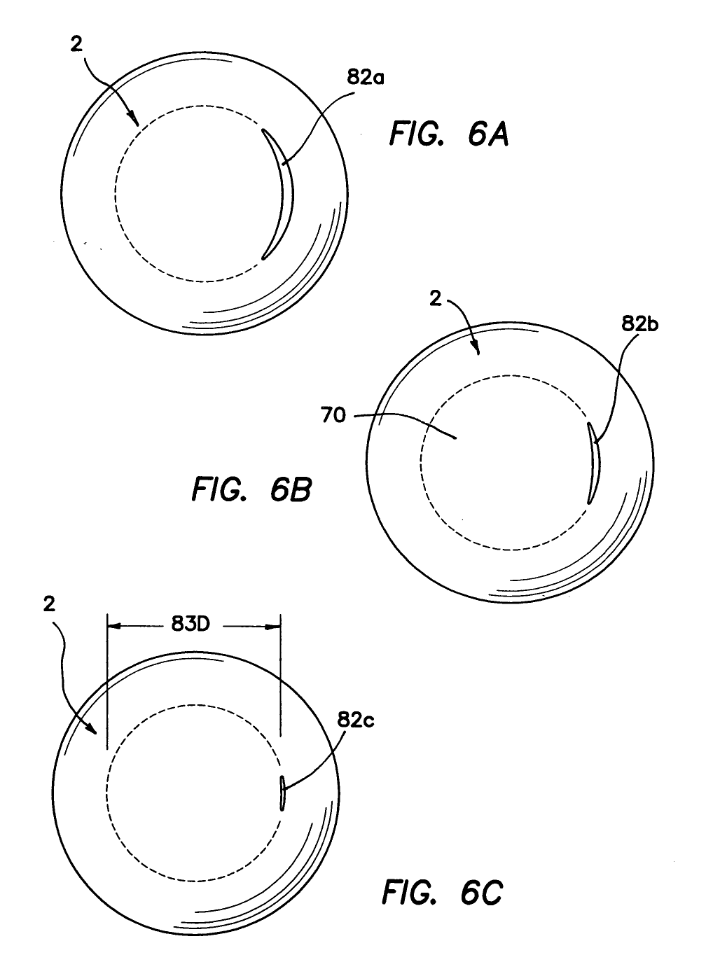

Incisions 82a, 82b, 82c of different sizes may be formed in accordance with the present invention, such as shown, for example in FIGS. 6A-6C respectively. The pocket diameter may also vary depending upon the size and shape of the lens to be implanted. For example, a relatively small incision 82c as shown in FIG. 6C may be used to provide a relatively large pocket diameter 83D. The incision 82c may be 1 mm in length, and may accommodate a lens in a deformed, such as a folded or rolled, configuration.

Additionally or alternatively, the incision size may be varied to accommodate various insertion techniques, such as whether a lens is deformed prior to insertion. Thus, a large incision may be formed when a lens is inserted in a substantially undeformed state, or a small incision may be formed when a lens is inserted in a deformed state. A large incision may be about 6 mm long when the lens has a diameter of about 6 mm.

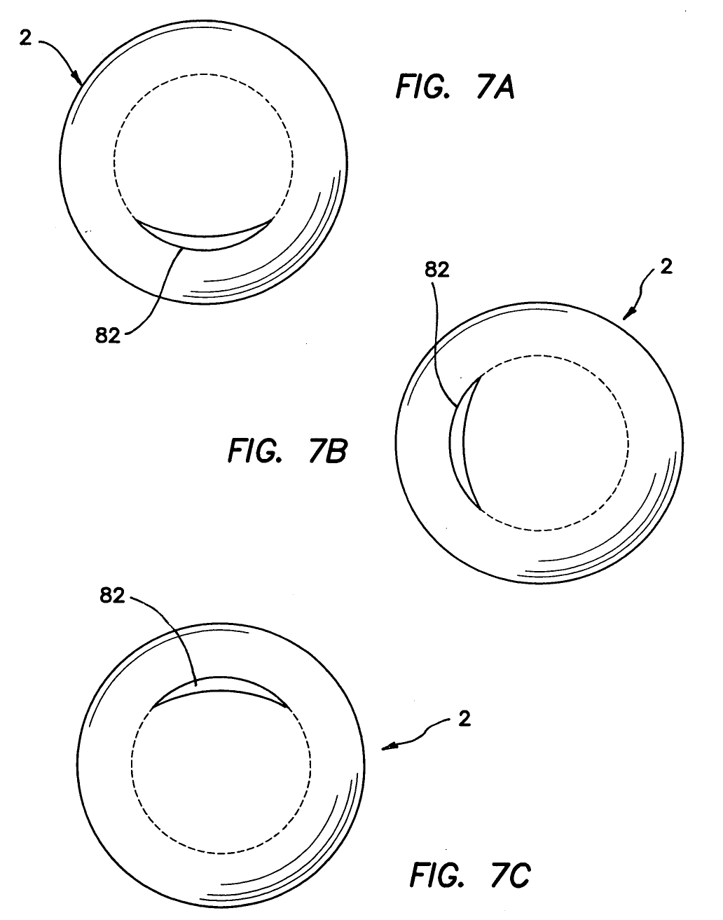

Turning now to FIGS. 7A-7C, it is shown that an initial slit or incision 82 may be formed at any desired region around the epithelium, but in preferred embodiments, the incision or incisions is formed either in the temporal portion of the cornea (e.g., the portion of the cornea that is located away from the nose of a patient), a nasal portion, a superior portion, or an inferior portion. It is to be appreciated that in some instances, it may be desirable to form the incision in the medial (generally central) portion of the cornea. In any event, the incision is preferably formed to provide an opening in the cornea, for example, of suitable size, to accommodate a corrective ocular device to be inserted therethrough without creating flap.

The ocular device or lens may be inserted through the incision and into the pocket by using forceps, or other similar device.

In certain embodiments, it is desirable to form a relatively small incision, such as the incision shown in FIG. 6C, and deforming the ocular device prior to insertion through the incision so that the deformed ocular device is inserted through the incision and allowed to unroll, uncoil, or otherwise regain its undeformed, native configuration within the pocket. In other words, after being placed into the pocket within the stroma, the deformed ocular device can assume its native or original configuration (e.g., the configuration of the ocular device before being deformed). The lens 40 may then be “rolled”, as shown in FIG. 9A, or “folded”, as shown in FIG. 9B so that the lens 40 can be inserted in the incision 82. For example, the lens 40 shown in FIG. 9B is folded along its midline so that two substantially equal-sized portions overlap.

The deformed lens may then be inserted into the incision 82, utilizing a suitable introducer device. The introducer or inserter may be configured to deform at least a portion of the ocular device so that the device can fit through the incision, for example, through a smaller incision that would be necessary if the ocular device was not deformed. For example, the ocular device may be folded or rolled or curled so that its cross-sectional area is reduced while it is being inserted beneath the epithelium, as discussed herein. The insertion device may be a syringe-like device which includes a body with a distal end dimensioned to pass the lens under the anterior corneal tissues of an eye. In certain situations, the insertion device may be identical to, similar to, or at least somewhat similar to well known and publicly available intraocular lens inserters.

The initial incision can be made by cutting or slicing the epithelium using a sharp instrument, such as a microkeratome and the like, including the microkeratome disclosed hereinabove. Alternatively, or in addition, the incision can be made by using blunt dissection to create an opening in the stroma without cutting or slicing the structures. Blunt dissection provides an advantage of reduced injury to the epithelial cells and/or epithelial tissue.

To perform blunt dissection to form the pocket, a blunt shaped instrument may be used that has a thickness that reduces the potential for tearing the stromal layers as adjacent layers are being separated from one another. One suitable blunt dissector includes a plate, a wire, or a knife with a dull edge. A spatula is also a suitable blunt dissection apparatus. The blunt dissector is inserted under the anterior corneal tissues and is gently urged across the underlying surface to “tease” the adjacent tissue layers apart. The separation appears to follow a path of least resistance to provide a substantially complete separation without damaging either the epithelium, Bowman's membrane or the underlying stromal tissues. Separation proceeds across the surface of the cornea to obtain a void sized to accommodate a corrective ocular device.

Advantageously, the methods of the present invention provide means for correcting vision without permanently removing substantial portions of the stromal bed. The methods provide long-term vision correction that can be reversed, as opposed to procedures that permanently alter the shape of a patient's cornea, such as LASEK and LASIK procedures. In that regard, the inlay 40 may be removed from the patient if complications develop or the patient's vision changes. Thus, the present methods provide for long-term, but reversible, vision correction.

By way of example, and not by way of limitation, a procedure for improving or enhancing or correcting a patient's vision may begin by a patient with a vision defect visiting a physician. After a comprehensive eye exam, a lens is prescribed and developed that will provide correction or improvement of the patient's vision. The lens may or may not be treated or modified to promote attachment of the lens to the stromal tissue. The patient returns to the physician's office for the procedure. An incision is made along an edge of the cornea and into the stroma. By utilizing the incision to enter the stroma, the physician forms a pocket between layers of the stroma using a blade, laser or other means to form a separation between stromal layers that will accommodate the lens to be inserted, without increasing the size of the initial incision. Preferably, the pocket between the stromal layers is formed so that the diameter of the pocket within the cornea substantially corresponds to the diameter of the lens. The incision is allowed to heal. The patient reports improvement in vision. The patient returns to the physician ten months after the procedure and upon examination of the treated eye, it is determined that the lens has not migrated within the eye and has not become decentered.

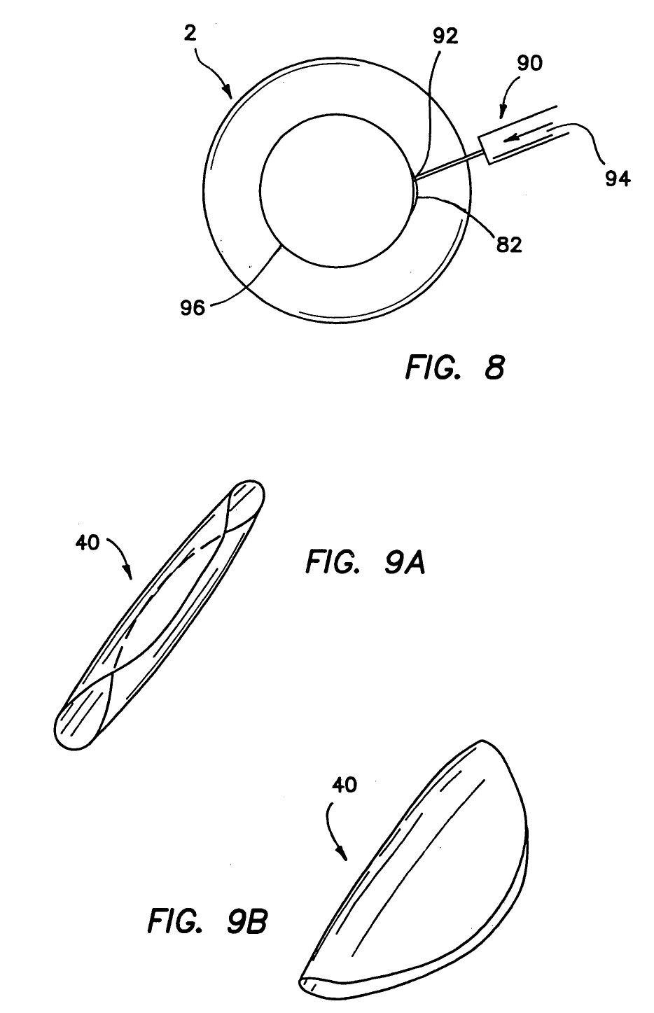

In certain embodiments, concurrent with or subsequent to the forming of the initial incision, a portion of the epithelium is raised using a vacuum. The vacuum may be provided with a microkeratome, such as with the separator disclosed in U.S. Patent Publication Nos. 2003/0018347 and 2003/0018348, or it may be provided as a separate instrument. Alternatively, or in addition, the stromal pocket may be formed by delivering a fluid between the stromal layers to form a fluid filled bleb. For example, a small incision 82 may be made in the epithelium of an eye, as shown in FIG. 8. A syringe device 90 having a distal end 92 and a fluid 94 located in the body of the syringe device 90 may be placed in proximity to the eye 2 so that the distal end 92 can pass the fluid 94 beneath the epithelium and Bowman's layer, and into the stromal tissues of the eye 2, as shown in FIG. 8. The fluid 94 causes adjacent layers of stromal tissue to separate and form bleb 96, as shown. A lens may then be placed therein and the fluid is allowed to flow out of the incision and decrease in volume. One suitable fluid may include sodium chloride, for example, an aqueous sodium chloride solution. Another fluid may include a gel. The gel may be a gel that includes at least one water soluble or water swellable polymeric material, for example, at least one cellulosic component, such as hydroxymethylcellulose and the like, and/or one or more other water soluble or water swellable polymeric materials. In one specific embodiment, the fluid comprises a gel sold as GENTEAL gel by CibaVision, Duluth, Ga.

One or more incisions may be made in the corneal tissue using a cutting procedure or blunt dissection procedures, as discussed above. Importantly, in this aspect of the invention, the cornea is cut without forming a corneal flap. In addition, the ocular device is inserted beneath the epithelium substantially without uncovering or exposing an anterior surface of Bowman's membrane. In practicing this method of the invention, the stroma of the cornea is preferably maintained in a substantially intact or undamaged state.

The foregoing methods may also include a step of applying a healing agent to the eye to promote a more rapid and effective healing of the cornea after insertion of the lens. In certain embodiments, the healing agent includes an antimicrobial, for example, selected from such materials which are conventional and/or well known for use in ophthalmic applications, to reduce potential contamination and infection. The healing agents may be any suitable ophthalmic composition which promotes cellular growth and/or reduces cellular death.

Still further in accordance with the invention disclosed herein, a reversible vision correction procedure has been invented. The method includes a step of inserting a corrective ocular device within a pocket of the stroma of a cornea of an eye, preferably substantially without forming a flap, and a step of removing the corrective ocular device from the eye. Among other things, if a patient finds that the corrective ocular device is or becomes insufficient to provide the desired vision correction, or is otherwise unsatisfactory in performance or comfort, the ocular device can be removed, and the patient's vision can be returned to its previous state. Thus, a patient can experience an improvement in vision similar to that provided by current LASIK and LASEK procedures, but with the advantage of being able to restore the patient's vision if the patient or physician is not completely satisfied with the vision correction.

The method may also include another step of inserting another corrective ocular device after the first ocular device is removed. For example, if the correction provided by the first ocular device is not sufficient to adequately improve the patient's vision, a second ocular device, for example, with different vision correcting properties, may be inserted to obtain the desired vision correction.

In practicing the foregoing methods, the corrective ocular device is preferably a vision correcting lens, however, other suitable devices that may augment the focusing capabilities of the eye may be utilized.

While this invention has been described with respect to various specific examples and embodiments, it is to be understood that the invention is not limited thereto and other embodiments are within the scope of the invention.

A number of publications and patents have been cited hereinabove. Each of the cited publications and patents are hereby incorporated by reference in their entireties.

What is claimed is:

1. A method for enhancing vision of an individual, comprising:
placing a vision correcting ocular device into a pocket formed in the stroma of an eye of an individual.

2. The method of claim 1, further comprising forming an incision in the stroma, and the placing step comprises inserting the ocular device through the incision.

3. The method of claim 2, wherein the incision is sized to accommodate the device in a deformed configuration.

4. The method of claim 2 wherein the incision is sized to accommodate the device in a non-deformed configuration.

5. The method of claim 2 wherein the step of forming an incision includes forming an incision in at least one of an approximate nasal portion of the cornea, an approximate temporal portion of the cornea, an approximate superior portion of the cornea, and an approximate inferior portion of the cornea.

6. The method of claim 2, wherein the incision has a length in a range of about 1 mm to about 6 mm.

7. The method of claim 1, further comprising deforming the ocular device prior to the placing step.

8. The method of claim 1, further comprising removing the ocular device from the eye, and placing another vision correcting ocular device into the stroma of the cornea of the eye.

9. The method of claim 1, wherein the ocular device is a vision correcting lens.

10. The method of claim 1, wherein the ocular device comprises a synthetic material.

11. The method of claim 1, wherein the ocular device comprises a synthetic polymeric material.

12. The method of claim 1, wherein the placing step occurs without forming a flap.

13. The method of claim 1, further comprising administering a healing agent to the eye in an amount effective to promote healing of the eye.

14. The method of claim 1, wherein the placing step includes forming a pocket by delivering a fluid into the cornea to separate tissues of the stroma.

15. The method of claim 1, wherein the placing step comprises using a blunt instrument to separate tissues of the stroma.

16. The method of claim 15, wherein the blunt instrument is a spatula or a wire.

17. A method for enhancing vision of an individual, comprising
forming a pocket in a cornea of a person, the pocket having a peripheral edge and a pocket opening located substantially adjacent the peripheral edge; and
inserting a lens into the pocket by directing the lens in a deformed configuration through the pocket opening and allowing the lens to assume a non-deformed configuration in the pocket so that a peripheral edge of the lens is substantially adjacent to the peripheral edge of the pocket and the lens is maintained in a substantially fixed position in the pocket.

18. A method for enhancing vision of an individual, comprising:
forming a pocket in the stroma of an eye of a person by delivering fluid into the eye to separate stromal tissue and form a pocket therein; and
placing a vision correcting ocular device into the fluid-formed stromal pocket.
